(19) United States
(12) Patent Application Publication
(10) Pub. No.: US 2009/0043306 A1
(43) Pub. Date: Feb. 12, 2009

(54) Clavicle Clamp Mechanism

(76) Inventor: Keith Baumgarten, Sioux Falls, SD (US)

Correspondence Address: MERCHANT & GOULD PC, P.O. BOX 2903, MINNEAPOLIS, MN, 55402-0903, US

(21) Appl. No.: 11/834,161

(22) Filed: Aug. 6, 2007

(51) Int. Cl. A61B 17/00

(52) U.S. Cl. 606/57; 606/54

(57) ABSTRACT

A clavicle clamp mechanism includes a first compression member, a second compression member, and an angular guide having a profile with a first end portion and a second end portion. The first compression member is disposed along the profile of the angular guide and is selectively rotatable about the angular guide. The second compression member is disposed along the profile of the angular guide between the first compression member and the second end portion. The second compression member defines a central axis and is selectively moveable along its central axis.

### BACKGROUND

An acromioclavicular separation injury (“AC separation”) is a common injury to the acromioclavicular joint of the shoulder that typically results from a fall on an outstretched hand or from sudden and direct contact with the shoulder such as that received through sports like football, rugby, or hockey. AC separation is categorized into one of six types (types I-VI), with the latter types being the most severe.

In types I-III, the ligaments that stabilize the clavicle are either sprained or partially severed. However, the clavicle is not significantly displaced from the scapula. These types of injuries can often heal through non-surgical treatments such as immobilization and anti-inflammatory drugs.

In types IV-VI, the ligaments that stabilize the clavicle are completely severed. In addition, the clavicle is vertically displaced from the scapula with the severity of the vertical displacement increasing as the type category increases. AC separation characterized as type IV-VI often requires surgical fixation, repair or reconstruction.

While there are a number of conventional surgical procedures for fixing, repairing or reconstructing the ligaments of the acromioclavicular joint, a need presently exists for reducing the vertical displacement of the clavicle with respect to the scapula prior to the performing those surgical procedures.

### SUMMARY

An aspect of the present disclosure relates to a clavicle clamp mechanism. The clavicle clamp mechanism includes an angular guide having a profile, a first end portion, and a second end portion. The clavicle clamp mechanism further includes a first compression member, and a second compression member. The first compression member is disposed along the profile of the angular guide and is selectively rotatable about the angular guide. The second compression member is disposed along the profile of the angular guide between the first compression member and the second end portion. The second compression member defines a central axis and is selectively moveable along the central axis of the second compression member.

Another aspect of the present disclosure relates to a clavicle clamp configured to vertically orient a clavicle with respect to a scapula during repair of an acromioclavicular separation injury. The clavicle clamp includes an angular guide, a first compression member and a second compression member. The angular guide of the clavicle clamp has an arcuate profile, a first end portion, and a second end portion. The first compression member is disposed along the arcuate profile of the angular guide and includes a collar that is selectively rotatable about the angular guide and a pin member that is coupled to the collar. The second compression member includes a threaded rod and defines a central axis. The second compression member is in threaded engagement with the angular guide and is selectively moveable along the central axis of the second compression member.

Another aspect of the present disclosure relates to a method of using a clavicle clamp mechanism. The method includes placing an engaging surface of a second compression member on a superior surface of a displaced clavicle of an injured shoulder. The first compression member is rotated and translated about and along an angular guide until the first compression member is secured to a scapula of the injured shoulder. An actuation end portion of the second compression member is actuated so as to reduce the displacement of the clavicle with respect to the scapula.

A variety of additional aspects will be set forth in the description that follows. These aspects can relate to individual features and to combinations of features. It is to be understood that both the foregoing general description and the following detailed description are exemplary and explanatory only and are not restrictive of the broad concepts upon which the embodiments disclosed herein are based.

### DESCRIPTION OF THE DRAWINGS

FIG. 1 is an anterior view of a right shoulder.

FIG. 2 is an anterior view of the right shoulder of FIG. 1 with an acromioclavicular separation injury.

FIG. 3 is a perspective view of a clavicle clamp mechanism having features that are examples of aspects in accordance with the principles of the present disclosure.

FIG. 4 is a cross-sectional view of the clavicle clamp mechanism of FIG. 3.

FIG. 5 is a right side view of an alternate embodiment of a clavicle clamp mechanism having features that are examples of aspects in accordance with the principles of the present disclosure.

FIG. 6 is an anterior view of the right shoulder of FIG. 2 with the clavicle clamp mechanism of FIG. 3 in an exemplary position with respect to the right shoulder.

### DETAILED DESCRIPTION

Reference will now be made in detail to the exemplary aspects of the present disclosure that are illustrated in the accompanying drawings. Wherever possible, the same reference numbers will be used throughout the drawings to refer to the same or like structure.

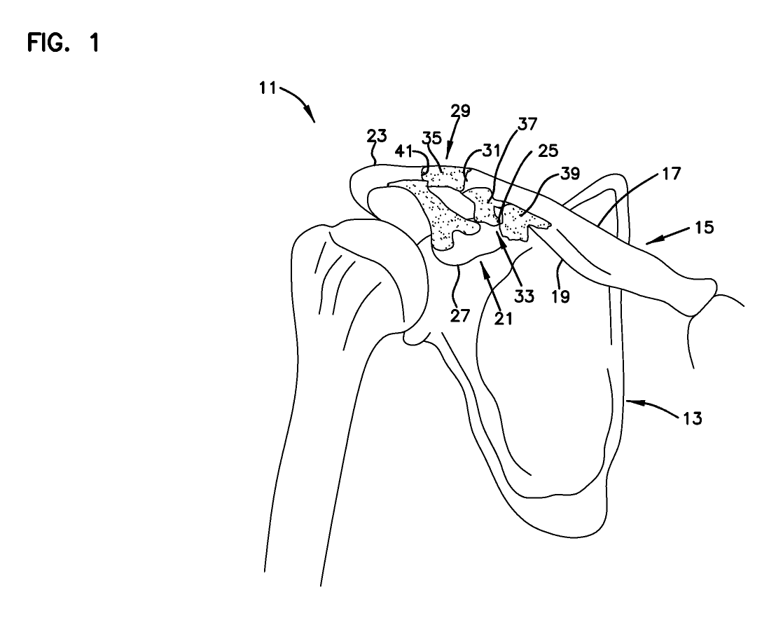

Referring now to FIG. 1, an anterior view of an uninjured right shoulder, generally designated 11, is shown. The shoulder 11 includes a scapula, generally designated 13, and a clavicle, generally designated 15, having a superior surface 17 and an inferior surface 19. The scapula 13 includes a coracoid process, generally designated 21, and an acromion 23. The coracoid process 21 is disposed on an upper portion of the anterior side of the scapula 13 and extends forwardly and laterally with respect to the scapula 13. The coracoid process 21 includes a superior side 25 and an inferior side 27. The acromion 23 is disposed on an upper portion of the posterior side of the scapula 13 and extends rearwardly and laterally with respect to the scapula 13.

The shoulder 11 further includes an acromioclavicular joint (“AC joint”), generally designated 29, disposed at the top of the shoulder 11 between the acromion 23 and a distal end 31 of the clavicle 15. The AC joint 29 is a gliding joint that allows the arm to be raised above the head. The AC joint 29 is stabilized by a plurality of ligaments including coracoclavicular ligaments, generally designated 33, and an acromioclavicular ligament 35.

The coracoclavicular ligaments 33 are attached to the superior side 25 of the coracoid process 21 and the inferior surface 19 of the clavicle 15 such that the coracoclavicular ligaments 33 connect the clavicle 15 to the coracoid process 21. The coracoclavicular ligaments 33 include a trapezoid ligament 37 and a conoid ligament 39, which is disposed posteriorly and medially to the trapezoid ligament 37.

The acromioclavicular ligament 35 is attached to a medial side 41 of the acromion 23 and the distal end 31 of the clavicle 15 such that the acromioclavicular ligament 35 extends between the acromion 23 and the clavicle 15.

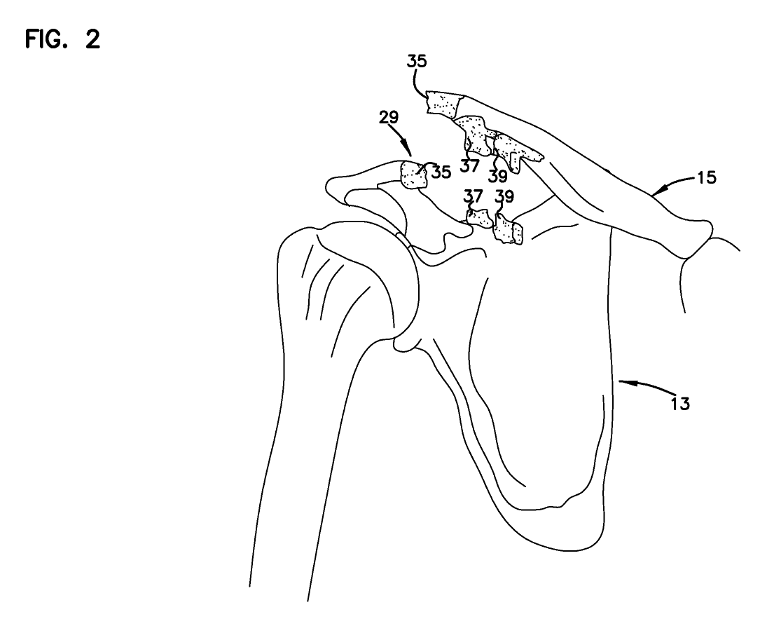

Referring now to FIG. 2, the shoulder 11 is shown with a separation injury at the AC joint 29 (“AC separation injury”). AC separation injuries are categorized into six types (Type I-Type VI). In types IV through VI, the acromioclavicular ligament 35, the trapezoid ligament 37, and the conoid ligament 39 are torn and the clavicle 15 is vertically displaced from the scapula 13 with the severity of the vertical displacement increasing as the type category increases. A type V AC separation injury is shown in FIG. 2. This type of injury requires surgical fixation, repair, or reconstruction of the coracoclavicular ligaments 33 in order to stabilize the clavicle 15.

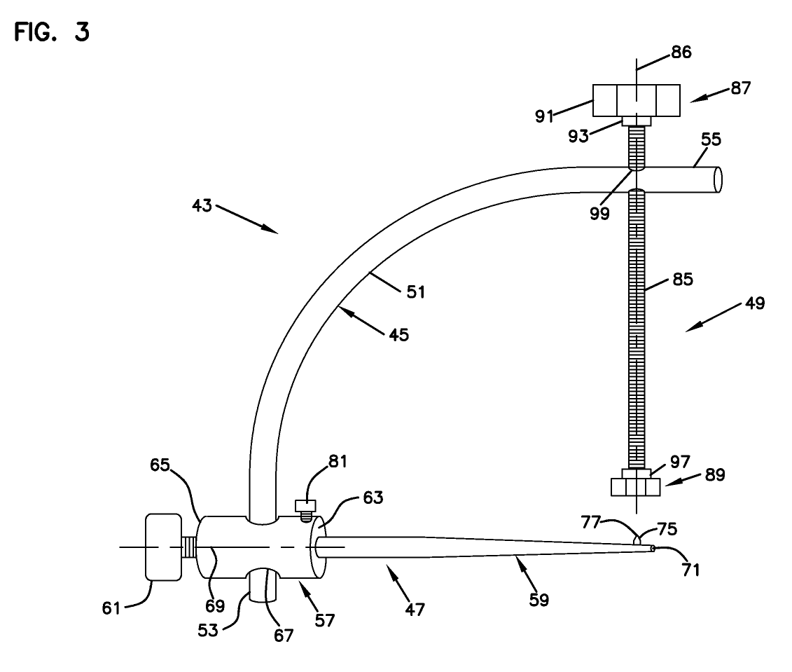

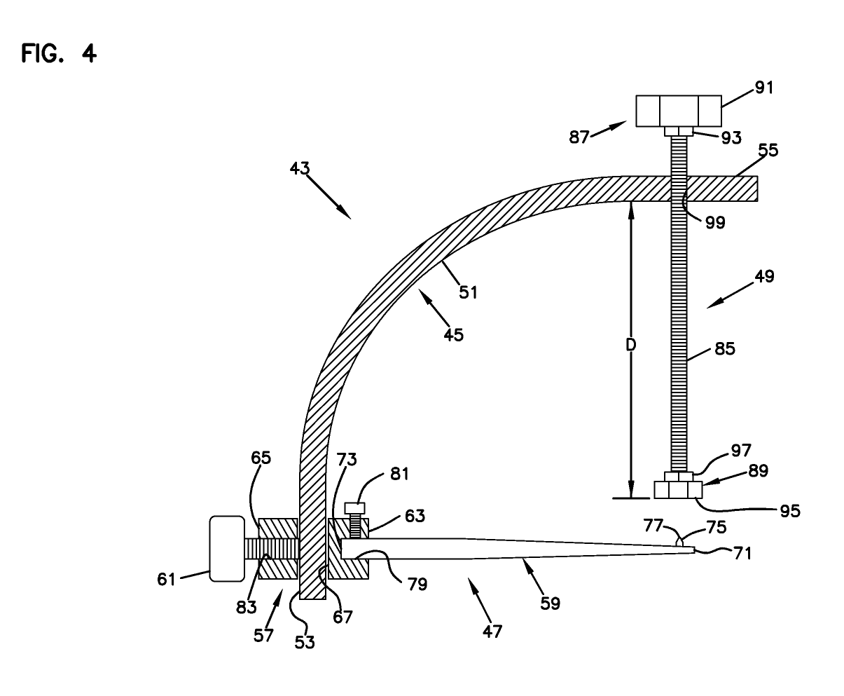

Referring now to FIGS. 3 and 4, a clavicle clamp mechanism, generally designated 43, which is used for reducing the vertical displacement between the clavicle 15 and the scapula 13 of the injured shoulder 11 prior to surgical fixation, repair, or reconstruction of the coracoclavicular ligaments 33, will now be described. The clavicle clamp mechanism 43 includes an angular guide, generally designated 45, a first compression member, generally designated 47, and a second compression member, generally designated 49.

In the subject embodiment, the angular guide 45 includes a rod 51 having a circular cross-section and an arcuate profile. It will be understood, however, that the scope of the present disclosure is not limited to the rod 51 of the angular guide 45 having a circular cross-section or an arcuate profile. The angular guide 45 further includes a first end portion 53 and a second end portion 55. In the subject embodiment, the first end portion 53 and the second end portion 55 are coplanar with the first end portion 53 being at an orientation that is approximately 90 degrees with respect to the orientation of the second end portion 55. It will be understood, however, that the scope of the present disclosure is not limited to the first end portion 53 and the second end portion 55 being coplanar or to the first end portion 53 being at a 90 degree orientation with respect to the second end portion 55.

The first compression member 47 includes a collar, generally designated 57, a pin member, generally designated 59, and a set screw 61. In the subject embodiment, the collar 57 is cylindrical in shape. It will be understood, however, that the scope of the present disclosure is not limited to the collar 57 being cylindrical in shape. The collar 57 includes a first axial end 63 and a second axial end 65. The collar 57 defines a thru-hole 67, which in the subject embodiment is generally perpendicular to a center axis 69 (shown as a dashed line in FIG. 3) through the first and second axial ends 63, 65 of the collar 57. The thru-hole 67 is sized slightly larger than the rod 51 of the angular guide 45 to allow for movement of the collar 57 with respect to the angular guide 45. While the collar 57 is shown disposed at the first end portion 53 of the angular guide 45, the collar 57 may be selectively moved to any location along the profile of the angular guide 45 between the first end portion 53 and the second compression member 49. In addition to being selectively moveable along the profile of the angular guide 45, the collar 57 can be selectively rotated about the rod 51 of the angular guide 45.

In the subject embodiment, the pin member 59 is in connected engagement with the first axial end 63 of the collar 57 so as to be generally perpendicular to the angular guide 45 at the location of the collar 57. The pin member 59 includes a first end 71 and a second end 73 (shown in FIG. 4). In the subject embodiment, the shape of the pin member 59 is generally cylindrical with a slight taper such that the diameter of the first end 71 is smaller than the diameter of the second end 73. It will be understood, however, that the scope of the present disclosure is not limited to the pin member 59 having a tapered cylindrical shape. A gripping surface 75 is provided near the first end 71 of the pin member 59. In the subject embodiment, the gripping surface 75 is a stud having a pointed end 77. The function of the gripping surface 75 will be described subsequently.

The second end 73 of the pin member 59 is in connected engagement with a mounting hole 79 located on the first axial end 63 of the collar 57. In the subject embodiment, the pin member 59 is secured in the mounting hole 79 by a screw 81 that is threadedly engaged with the collar 57. The screw 81 selectively engages the second end 73 of the pin member 59 so as to bind the pin member 59 in the mounting hole 79. However, as the second end 73 of the pin member 59 could alternatively be threaded, press fit, bonded, or soldered into the mounting hole 79, the scope of the present disclosure is not limited to the screw 81 selectively engaging the second end 73 of the pin member 59.

The set screw 61 is in threaded engagement with a threaded hole 83 (shown only in FIG. 4) in the collar 57 that extends from the second axial end 65 of the collar 57 into the thru-hole 67. In the subject embodiment, the threaded hole 83 is generally perpendicular to the thru-hole 67. As previously described, the collar 57 can be selectively moved along the profile of the angular guide 45 between the first end portion 53 and the second compression member 49 and selectively rotated about the rod 51 of the angular guide 45. With the collar 57 in a desired position along and about the angular guide 45, the set screw 61 is tightened until the set screw 61 abuts the rod 51 of the angular guide 45, which is disposed in the thru-hole 67, so as to secure the collar 57 at both the desired position along the angular guide 45 and the desired orientation with respect to the angular guide 45.

The second compression member 49 includes a rod member 85 defining a central axis 86 (shown in FIG. 3) and having an actuation end portion 87 and an oppositely disposed engaging end portion 89. As will be described in greater detail subsequently, the second compression member 49 is selectively moveable along its central axis 86. In the subject embodiment, the second compression member 49 is selectively moveable through the actuation of the actuation end portion 87. To assist in actuation, the actuation end portion 87 includes an outer surface 91 having a hexagonal shape. The hexagonal shape allows the actuation end portion 87 to be actuated by hand, with a wrench or other tool. It will be understood, however, that as other shapes of the outer surface 91 would allow the actuation end portion 87 to be actuated by hand or with a tool, the scope of the present disclosure is not limited to the outer surface 91 having a hexagonal shape. In the subject embodiment, the actuation end portion 87 is in threaded engagement with the rod member 85. A nut 93 secures the actuation end portion 87 to the rod member 85.

The engaging end portion 89 includes an engaging surface 95, the purpose of which will be described in greater detail subsequently. In the subject embodiment, the engaging end portion 89 is in threaded engagement with the rod member 85. A nut 97 secures the engaging end portion 89 to the rod member 85.

In the embodiments of FIGS. 3 and 4, the rod member 85 is in threaded engagement with an aperture 99 through the second end portion 55 of the angular guide 45. The threaded engagement between the rod member 85 and the angular guide 45 allows the second compression member 49 to be selectively displaced along the central axis 86. By rotating the actuation end portion 87 of the second compression member 49, the axial displacement (shown as dimension D in FIG. 4) of the second compression member 49 can be selectively increased or decreased.

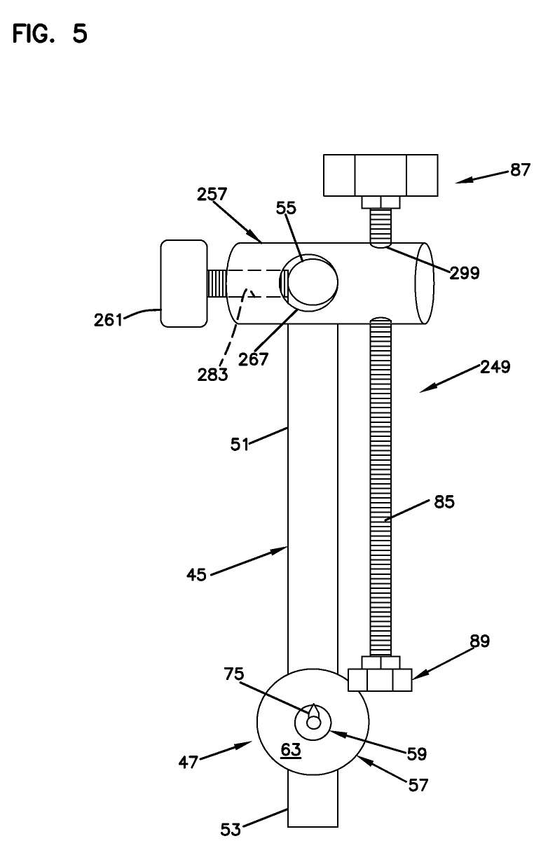

Referring now to FIG. 5, an alternate embodiment of a second compression member, generally designated 249, is shown. The alternate embodiment of the second compression member 249 includes the rod member 85 with the actuation end portion 87 and the oppositely disposed engaging end portion 89. In this alternate embodiment, the rod member 85 is in threaded engagement with a collar member, generally designated 257.

The collar member 257 includes a set screw 261 and defines a thru-hole 267 and a threaded aperture 299. In the alternate embodiment of the second compression member 249, the thru-hole 267 is sized slightly larger than the rod 51 of the angular guide 45 to allow for movement of the collar member 257 with respect to the angular guide 45. While the collar member 257 is shown disposed at the second end portion 55 of the angular guide 45, the collar member 257 may be selectively moved to any location along the profile of the angular guide 45 between the second end portion 55 and the first compression member 47. In addition, the collar member 257 can be selectively rotated about the rod 51 of the angular guide 45.

The set screw 261 is in threaded engagement with a threaded hole 283 (shown as dashed lines in FIG. 5) in the collar member 257 that extends through the collar member 257 and into the thru-hole 267. In the subject embodiment, the threaded hole 283 is generally perpendicular to the thru-hole 267. With the collar member 257 in a desired position along and about the angular guide 45, the set screw 261 is tightened until the set screw 261 abuts the rod 51 of the angular guide 45, which is disposed in the thru-hole 267, so as to secure the collar member 257 in the desired position.

In the subject embodiment, the components of the clavicle clamp mechanism 43 are manufactured from a stainless steel material. However, due to the variety of different manufacturing materials of which the components of the clavicle clamp mechanism 43 could be manufactured, it will be understood that the scope of the present disclosure is not limited to the material of the clavicle clamp mechanism 43 being stainless steel.

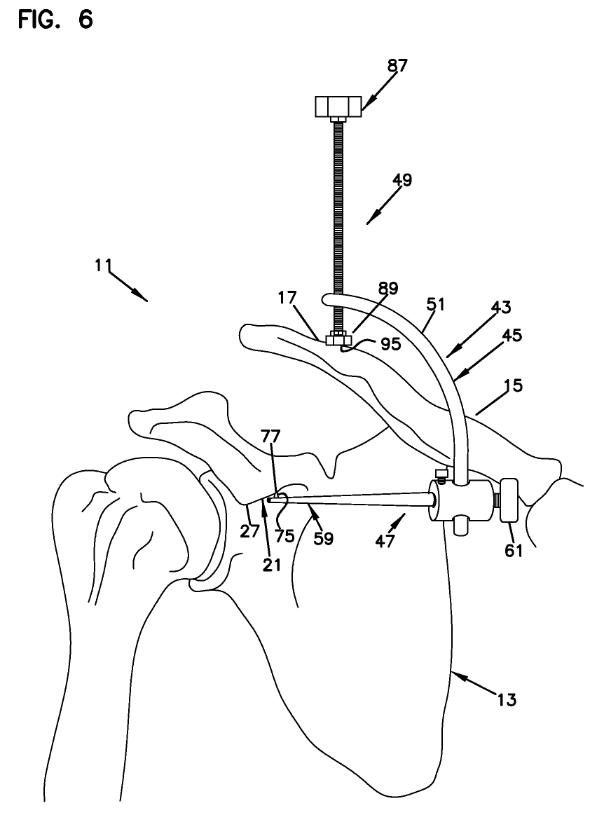

Referring now to FIG. 6, a method for using the clavicle clamp mechanism 43 will be described. The engaging surface 95 of the engaging end portion 89 of the second compression member 49 is placed on the superior surface 17 of the vertically displaced clavicle 15. With the engaging surface 95 placed on the superior surface 17 of the clavicle 15, the second compression member 49 is oriented such that increasing the distance D (shown in FIG. 3) of the clavicle clamp mechanism 43 decreases the vertical displacement of the clavicle 15 with respect to the scapula 13.

With the second compression member 49 positioned on the superior surface 17 of the clavicle 15, the first compression member 47 is translated along the angular guide 45 and rotated about the rod 51 of the angular guide 45 until the gripping surface 75 of the pin member 59 is engaged with the inferior side 27 of the coracoid process 21. By engaging the gripping surface 75 of the clavicle clamp mechanism 43 and the inferior side 27 of the coracoid process 21, the clavicle clamp mechanism 43 is secured or anchored to the shoulder 11. The anchoring of the clavicle clamp mechanism 43 to the scapula 13 of the shoulder 11 allows for the engaging surface 95 of the second compression member 49 to directly transmit the movement of the second compression member 49 to the vertically displaced clavicle 15. With the first compression member 47 engaged to the inferior side 27 of the coracoid process 21, the set screw 61 is tightened to retain the first compression member 47 in its position along the angular guide 45.

As previously described, the gripping surface 75 of the first compression member 47 in the subject embodiment is a stud having a pointed end 77. The pointed end 77 of the gripping surface 75 engages the inferior side 27 of the coracoid process 21 and reduces the risk of the first compression member 47 becoming disengaged with the coracoid process 21 during use of the clavicle clamp mechanism 43. It will be understood, however, that the scope of the present disclosure is not limited to the gripping surface 75 having a pointed end 77.

With the first compression member 47 securing the clavicle clamp mechanism 43 to the shoulder 11 and the second compression member 49 disposed on the vertically displaced clavicle 15, the actuation end portion 87 of the second compression member 49 can be actuated to reduce the vertical displacement of the clavicle 15. As previously described, the actuation end portion 87 is actuated by rotating the actuation end portion 87 in either the clockwise or counterclockwise direction. In order to reduce the vertical displacement of the clavicle 15 with respect to the scapula 13, the actuation end portion 87 should be rotated such that the distance D (shown in FIG. 3) between the angular guide 45 and the engaging surface 95 increases.

When the vertical distance between the clavicle 15 and the scapula 13 is reduced to an acceptable amount, conventional fixation, repair, or reconstruction techniques can be used to stabilize the clavicle 15. After the clavicle 15 is stabilized, the clavicle clamp mechanism 43 can be removed by rotating the actuation end portion 87 of the second compression member 49 such that the distance D decreases and by disengaging the first compression member 47 from the inferior surface 27 of the coracoid process 21.

While the clavicle clamp mechanism 43 and the method for using the clavicle clamp mechanism 43 has been described with regard to an acromioclavicular joint separation injury, it will be understood that the scope of the present disclosure is not limited to acromioclavicular joint separation injuries as the present disclosure could be used with other injuries, such as distal clavicle fractures.

Various modifications and alterations of this disclosure will become apparent to those skilled in the art without departing from the scope and spirit of this disclosure, and it should be understood that the inventive scope of this disclosure is not to be unduly limited to the illustrative embodiments set forth herein.

What is claimed is:

1. A clavicle clamp mechanism, comprising
an angular guide having a profile, wherein the angular guide includes a first end portion and a second end portion;
a first compression member disposed along the profile of the angular guide, wherein the first compression member is selectively rotatable about the angular guide; and
a second compression member disposed along the profile of the angular guide between the first compression member and the second end portion and defining a central axis, wherein the second compression member is selectively moveable along the central axis.

2. A clavicle clamp mechanism as claimed in claim 1, wherein the profile of the angular guide is arcuate.

3. A clavicle clamp mechanism as claimed in claim 1, wherein the first compression member is selectively moveable along the profile of the angular guide.

4. A clavicle clamp mechanism as claimed in claim 1, wherein the first compression member includes a collar disposed along the angular guide.

5. A clavicle clamp mechanism as claimed in claim 4, wherein the collar is selectively secured to the angular guide through a set screw.

6. A clavicle clamp mechanism as claimed in claim 4, wherein the first compression member further includes a pin member coupled to the collar, the pin member having a gripping surface.

7. A clavicle clamp mechanism as claimed in claim 6, wherein a screw couples the pin member to the collar.

8. A clavicle clamp mechanism as claimed in claim 6, wherein the gripping surface is a stud having a pointed end.

9. A clavicle clamp mechanism as claimed in claim 6, wherein the pin member extends from the collar in a generally perpendicular direction from the angular guide.

10. A clavicle clamp mechanism as claimed in claim 1, wherein the second compression member includes a threaded rod having an actuation end portion and an oppositely disposed engaging end portion.

11. A clavicle clamp mechanism as claimed in claim 10, wherein the threaded rod of the second compression member is in threaded engagement with a threaded aperture defined by the second end portion of the angular guide.

12. A clavicle clamp mechanism as claimed in claim 10, wherein the threaded rod of the second compression member is in threaded engagement with a threaded aperture defined by a collar member coupled to the angular guide.

13. A clavicle clamp mechanism as claimed in claim 12, wherein the collar member is disposed along the profile of the angular guide and selectively rotatable about the angular guide.

14. A clavicle clamp mechanism as claimed in claim 13, wherein the collar member of the second compression member is selectively moveable along the profile of the angular guide.

15. A clavicle clamp configured to vertically orient a clavicle with respect to a scapula during repair of an acromioclavicular separation injury, the clavicle clamp comprising
an angular guide having an arcuate profile, wherein the angular guide includes a first end portion and a second end portion;
a first compression member disposed along the profile of the angular guide, wherein the first compression member includes a collar that is selectively rotatable about the angular guide and a pin member that is coupled to the collar; and
a second compression member including a threaded rod and defining a central axis, wherein the second compression member is in threaded engagement with the angular guide and is selectively moveable along the central axis.

16. A clavicle clamp as claimed in claim 15, wherein the pin member of the first compression member includes a gripping surface.

17. A clavicle clamp as claimed in claim 15, wherein the first compression member is selectively moveable along the angular guide.

18. A method for using a clavicle clamp mechanism, comprising:
placing an engaging surface of a second compression member of the clavicle clamp on a superior surface of a displaced clavicle of an injured shoulder;
rotating and translating a first compression member about and along an angular guide of the clavicle clamp until the first compression member is secured to a scapula of the injured shoulder; and
actuating an actuation end portion of the second compression member so as to reduce displacement of the clavicle with respect to the scapula.

19. A method for using a clavicle clamp mechanism as claimed in claim 18, wherein the first compression member includes a collar that is selectively rotatable about the angular guide and a pin member that is connectedly engaged with the collar.

20. A method for using a clavicle clamp mechanism as claimed in claim 18, wherein the first compression member is secured to a coracoid process disposed on the scapula.
